(19) United States
(12) Patent Application Publication
(10) Pub. No.: US 2026/0345445 A1
(43) Pub. Date: Nov. 5, 2026

(54) OIL-IN-WATER TYPE EYELASH COSMETIC

(71) Applicant: JO COSMETICS CO., LTD., Ota-ku, Tokyo (JP)

(72) Inventors: Kaede SUDO, Tokyo (JP); Hiroka WATANABE SHIRAHATA, Tokyo (JP)

(73) Assignee: JO COSMETICS CO., LTD., Ota-ku, Tokyo (JP)

(21) Appl. No.: 19/483,459

(22) PCT Filed: Apr. 30, 2024
(86) PCT No.: PCT/JP2024/016681

§ 371 Date: Nov. 11, 2025

(30) Foreign Application Priority Data
May 16, 2023 (JP) 2023-080861

(51) Int. Cl. A61K 8/06; A61K 8/891; A61K 8/895; A61K 8/92; A61Q 1/10

(52) U.S. Cl. CPC A61K 8/062; A61K 8/891; A61K 8/895; A61K 8/922; A61Q 1/10; A61K 2800/5922

(57) ABSTRACT

An oil-in-water type cosmetic composition for eyelashes that exhibits high curling and volumizing effects while also having excellent separation properties comprises: (A) 1 to 12% by mass of an oil-soluble resin; (B) 3 to 10% by mass of a solid oil having a melting point of 50 to 120 °C; (C1) a low-melting-point silicone wax having a melting point in the range of 10 °C to less than 50 °C; and (C2) a high-melting-point silicone wax having a melting point of 50 to 110 °C, wherein the total amount of components (C1) and (C2) is 1 to 10% by mass, the mass ratio of component (C1) to component (C2), (C1)/(C2), is in the range of 1 to 5, and the mass ratio of the total amount of components (A) and (B) to the total amount of components (C1) and (C2), [(A)+(B)]/[(C1)+(C2)], is 2 or more.

### TECHNICAL FIELD

The present invention relates to an oil-in-water type cosmetic composition for eyelashes, and more particularly, to an oil-in-water type cosmetic composition for eyelashes that provides high curling and volumizing effects, while also exhibiting excellent eyelash separation.

### BACKGROUND ART

Cosmetic compositions for eyelashes, such as mascara, have cosmetic effects such as curling the eyelashes upward and making them appear thicker to enhance the appearance of the eyes. Conventionally, various formulations of these cosmetics for eyelashes have been known, such as oil-based types, water-in-oil or oil-in-water emulsion types, and aqueous types. Recently, oil-in-water emulsion mascaras containing film-forming polymers, which can be easily removed with warm water without the use of a dedicated, remover have become popular.

Mascaras are required to provide the basic performance of cosmetic effects such as curling eyelashes upward and making them appear thicker to enhance the appearance of the eyes. To enhance these effects, oil-soluble resins and high melting point waxes are added. However, especially in oil-in-water type cosmetic compositions for eyelashes, high concentrations of these ingredients tend to cause the eyelashes to bunch together, resulting in impaired eyelash separation and a tendency for clumping to occur, making it difficult to achieve both high curling and volumizing effects while maintaining eyelash separation.

For example, Patent Document 1 discloses an oil-in-water type cosmetic composition for eyelashes comprising a water-soluble polymer having stringiness properties and a low-melting-point solid/paste-like long-chain (alkyl-modified/alkoxy-modified) organopolysiloxane; however, the curling power and eyelash separation are not necessarily sufficient.

Patent Document 2 also discloses cosmetics comprising a combination of a high-melting-point silicone wax and a low-melting-point silicone wax. However, the curling and volumizing effects are not sufficient.

Furthermore, Patent Document 3 discloses an oil-in-water type cosmetic composition for eyelashes comprising a combination of candelilla resin and a low-melting-point solid/paste-like long-chain (alkyl-modified/alkoxy-modified) organopolysiloxane; however, the eyelash separation is not sufficient.

### CITATION LIST

### Patent Literature

[PTL 1] JP 2008-088112 A
[PTL 2] JP 2005-145886 A
[PTL 3] JP 2007-262034 A

### SUMMARY OF THE INVENTION

### Technical Problem

In order to enhance the effects of oil-in-water type cosmetic compositions for eyelashes, oil-soluble resins and high-melting-point waxes are blended; however, when blended at high concentrations, the eyelashes tend to bunch together, resulting in impaired eyelash separation and a tendency for clumping to occur, and thus there has been a problem in that it is difficult to achieve both high curling and volumizing effects while maintaining eyelash separation.

### Solution to Problem

This invention solves the above-mentioned problems by formulating an oil-soluble resin, a high-melting-point wax, a low-melting-point silicone wax, and a high-melting-point silicone wax in specific proportions.

That is, the present invention is as described below.
[1] An oil-in-water type cosmetic composition for eyelashes, comprising:
(A) 1 to 12% by mass of oil-soluble resin,
(B) 3 to 10% by mass of a solid oil having a melting point of 50 to 120 °C;
(C1) a low-melting-point silicone wax having a melting point in the range of 10 °C to less than 50 °C; and
(C2) a high-melting-point silicone wax having a melting point of 50 to 110 °C, wherein:
the total amount of component (C1) and component (C2) is in the range of 1 to 10% by mass,
the mass ratio of component (C1) to component (C2), (C1)/(C2), is in the range of 1 to 5, and
the mass ratio of the total amount of component (A) and component (B) to the total amount of component (C1) and component (C2), [(A)+(B)]/[(C1)+(C2)], is 2 or more.
[2] The cosmetic composition according to [1], wherein the mass ratio of the total amount of components (A) and (B) to the total amount of components (C1) and (C2),
[(A)+(B)]/[(C1)+(C2)], is in the range of 2.5 to 4.5.
[3] The cosmetic composition according to [1], wherein the solid oil of component (B) having a melting point of 50 to 120 °C comprises carnauba wax.
[4] The cosmetic composition according to [3], wherein the amount of carnauba wax is more than 50% by mass of the total amount of the solid oil.

### Advantageous Effects of Invention

This invention makes it possible to provide an oil-in-water type cosmetic composition for eyelashes which exhibits a high curling effect and a volumizing effect, as required for an oil-in-water type cosmetic composition for eyelashes, and which further has excellent eyelash separation.

### DESCRIPTION OF EMBODIMENTS

As the oil-soluble resin of component (A) used in the present invention, there is no particular limitation as long as it is soluble in an oil component. Examples include rosin-based resins such as glyceryl hydrogenated rosinate and pentaerythrityl rosinate, candelilla resin, polyvinyl acetate resin, polyvinyl isobutyl ether, polyisobutylene, and the like. Preferably, glyceryl hydrogenated rosinate and candelilla resin may be mentioned. The oil-soluble resins of the present invention do not include silicone resins such as trimethylsiloxysilicate. These components (A) may be used alone or in combination of two or more.

The content of component (A) in the oil-in-water type cosmetic composition for eyelashes of the present invention is 1 to 12% by mass, preferably 3 to 7% by mass, and more preferably 4 to 6% by mass. When component (A) is 1% by mass or more, good curling and volumizing effects can be obtained, and when it is 12% by mass or less, appropriate eyelash separation can be achieved.

The solid oil having a melting point of 50 to 120 °C used as component (B) in the present invention is not particularly limited, but examples thereof may be mentioned, such as carnauba wax, bleached beeswax, beeswax, candelilla wax, whale wax, montan wax, rice bran wax, lanolin, Japan wax, hexyl laurate, hydrogenated lanolin, hydrogenated jojoba oil, hard lanolin, shellac wax, microcrystalline wax, paraffin wax, polyethylene wax, Fischer-Tropsch wax, hardened oil, hydrogenated castor oil, jojoba esters, stearyl alcohol, behenyl alcohol, and the like. Preferably, carnauba wax and bleached beeswax may be mentioned. These components (B) may be used alone or in combination of two or more.

The content of component (B) in the oil-in-water type cosmetic composition for eyelashes of the present invention is 3 to 15% by mass, preferably 5 to 12% by mass, and more preferably 3 to 10% by mass. When component (B) is at 3% by mass or more, adequate curling and volumizing effects can be obtained, and when it is 15% by mass or less, good eyelash separation can be achieved.

Among the solid oils used as component (B) having a melting point of 50 to 120 °C, carnauba wax is particularly preferred because it can effectively improve the curling effect. The content of carnauba wax in component (B) is preferably 50% by mass or more, and more preferably 60% by mass or more.

The silicone waxes used as component (C1) in this invention, which have a melting point in the range of 10 °C to less than 50 °C, are not particularly limited so long as they are solid or paste-like at 25 °C. Examples thereof include long-chain alkyl-modified acrylic-silicone graft copolymers, alkyl-modified dimethylpolysiloxane, alkyl-modified dimethicone copolyol, and the like. Further examples include (acrylates/stearyl acrylate/dimethicone methacrylate) copolymer, (stearoxymethicone/dimethicone) copolymer, stearyl dimethicone, bis-stearyl dimethicone. Preferably, (acrylates/stearyl acrylate/dimethicone methacrylate) copolymer may be mentioned.

The silicone waxes used as component (C2) in this invention, which have a melting point of 50 to 110 °C, are solid at 25 °C and are not particularly limited so long as their melting points fall within the specified range. Examples thereof may include alkyl (C30-45) methicone and alkyl (C30-45) dimethicone. Preferably, alkyl (C30-45) methicone may be mentioned.

The total amount of component (C1) and component (C2) in the oil-in-water type cosmetic composition for eyelashes of the present invention may be 1 to 10% by mass, preferably 2 to 8% by mass, more preferably 3 to 6% by mass.

The ratio of component (C1) to component (C2) (mass ratio, (C1)/(C2)) may be mentioned as preferably 1 to 5, and more preferably 1 to 3. When the value of ((C1)/(C2) (mass ratio)) is 5 or less, adequate eyelash separation can be achieved. And when the mass ratio is 1 or more, a good volumizing effect can be obtained.

In the present invention, the ratio of the total amount of component (A) and component (B) to the total amount of component (C1) and component (C2) (mass ratio, [(A)+(B)]/[(C1)+(C2)]) may be mentioned as 2 or more. Preferably, it may be mentioned as 2.5 to 4.5, and more preferably 2.9 to 3.5. When the value of ([(A)+(B)]/[(C1)+(C2)]) is 2 or more, a good volumizing effect can be obtained, and when it is 5 or less, good eyelash separation can be achieved.

In addition to components (A) to (C), water-soluble ingredients, oil-soluble ingredients, surfactants, excipients, film-forming polymers, etc. can be blended into the oil-in-water type cosmetic composition for eyelashes of the present invention to the extent that they produce the effects of the invention.

As water-soluble ingredients, there is no particular limitation so long as they are water or water-soluble components. Examples thereof may include alcohols such as ethyl alcohol and isopropyl alcohol; glycols such as propylene glycol, 1,3-butylene glycol, and dipropylene glycol; glycerols such as glycerin, diglycerin, and polyglycerin; sugars such as sorbitol, maltitol, sucrose, starch sugar, and lactitol; water-soluble polymers such as guar gum, sodium chondroitin sulfate, gum arabic, sodium alginate, carrageenan, methyl cellulose, hydroxyethyl cellulose, and carboxyvinyl polymer; salts such as sodium chloride, magnesium chloride, and sodium lactate; plant extracts such as aloe vera, witch hazel, hamamelis, cucumber, lemon, lavender, and rose; and water.

As oil-soluble ingredients, there is no particular limitation regarding their origin, such as animal oils, vegetable oils, or synthetic oils, or their properties, such as semi-solid oils or volatile oils, provided that they are other than components (A) to (C). Examples thereof may include hydrocarbons, fats and oils, ester oils, higher alcohols, oil-based gelling agents, and the like.

Specifically, examples include hydrocarbons such as liquid paraffin, squalane, petrolatum, and polybutene; fats and oils such as olive oil, castor oil, jojoba oil, mink oil, and macadamia nut oil; esters such as cetyl isooctanoate, isopropyl myristate, isopropyl palmitate, octyldodecyl myristate, glyceryl trioctanoate, polyglyceryl diisostearate, diglyceryl triisostearate, neopentyl glycol dioctanoate, cholesterol fatty acid esters, and N-lauroyl-L-glutamic acid di(cholesteryl, behenyl, octyldodecyl); higher alcohols that are liquid at 25 °C such as oleyl alcohol and isostearyl alcohol; silicones such as low-viscosity dimethylpolysiloxane, high-viscosity dimethylpolysiloxane, methylphenylpolysiloxane, decamethylcyclopentasiloxane, octamethylcyclotetrasiloxane, polyether-modified polysiloxanes, polyoxyalkylene-alkylmethylpolysiloxane-methylpolysiloxane copolymers, crosslinked organopolysiloxanes, and fluorine-modified polysiloxanes; fluorinated oils such as perfluorodecane, perfluorooctane, and perfluoropolyethers; oil-based gelling agents such as dextrin fatty acid esters, sucrose fatty acid esters, starch fatty acid esters, aluminum isostearate, and calcium stearate; and silicone resins such as trimethylsiloxysilicate, partially crosslinked organopolysiloxanes, tri (trimethylsiloxy) silylpropyl carbamoyl pullulan, polymethylsilsesquioxane, polypropylene silsesquioxane, fluorine-modified silicones, acryl-modified silicones, and silicone dendrimer-modified resin compounds.

As surfactants, there is no particular limitation, and examples include glycerin fatty acid esters and their alkylene glycol adducts, polyglycerin fatty acid esters and their alkylene glycol adducts, propylene glycol fatty acid esters and their alkylene glycol adducts, sorbitan fatty acid esters and their alkylene glycol adducts, sorbitol fatty acid esters and their alkylene glycol adducts, polyoxyalkylene fatty acid esters, sucrose fatty acid esters, polyoxyalkylene alkyl ethers, glycerin alkyl ethers, polyoxyethylene alkyl phenyl ethers, polyoxyethylene hydrogenated castor oil, alkylene glycol adducts of lanolin, polyoxyalkylene alkyl-modified silicones, polyether-modified silicones, and the like.

As anionic surfactants, examples include inorganic and organic salts of fatty acids such as stearic acid and lauric acid, alkylbenzenesulfonates, alkyl sulfonates, α-olefinsulfonates, dialkylsulfosuccinates, a-sulfonated fatty acids, acyl methyltaurates, N-methyl-N-alkyl taurates, polyoxyethylene alkyl ether sulfates, polyoxyethylene alkyl phenyl ether sulfates, alkyl phosphates, polyoxyethylene alkyl ether phosphates, polyoxyethylene alkyl phenyl ether phosphates, N-acyl amino acid salts, N-acyl-N-alkyl amino acid salts, O-alkyl substituted maleates, alkyl sulfosuccinates, and the like.

As cationic surfactants, examples include alkylamine salts, polyamine and alkanolamine fatty acid derivatives, alkyl quaternary ammonium salts, cyclic quaternary ammonium salts, and the like.

As amphoteric surfactants, amino acid-type or betaine-type surfactants having carboxylic acid, sulfate ester, sulfonate, or phosphate ester groups can be used, provided that they are safe for human use. Examples include N, N-dimethyl-N-alkyl-N-carboxymethylammonium betaine, N, N-dialkylaminoalkylene carboxylic acids, N, N, N-trialkyl-N-sulfoalkylene ammonium betaine, N,N-dialkyl-N, N-bis(polyoxyethylene sulfate) ammonium betaine, 2-alkyl-1-hydroxyethyl-1-carboxymethylimidazolinium betaine, lecithin, and the like.

As excipients, there is no particular limitation; for example, they are not particularly restricted by shape (plate, spindle, needle, fiber, etc.), particle size, or particle structure (porous, non-porous, etc.), and include inorganic powders, glittering powders, organic powders, pigmented powders, composite powders, and the like.

Specifically, inorganic powders include Prussian Blue, ultramarine, red iron oxide, yellow iron oxide, black iron oxide, titanium dioxide, zinc oxide, aluminum oxide, cerium oxide, silicon dioxide, magnesium oxide, zirconium oxide, magnesium carbonate, calcium carbonate, chromium oxide, chromium hydroxide, carbon black, aluminum silicate, magnesium silicate, aluminum magnesium silicate, mica, synthetic mica, synthetic sericite, sericite, talc, kaolin, silicon carbide, barium sulfate, bentonite, smectite, boron nitride, and the like. Glittering powders include bismuth oxychloride, titanium dioxide-coated mica, iron oxide-coated mica, iron oxide-coated (titanium dioxide-coated mica), and organic pigment-treated (titanium dioxide-coated mica), aluminum powder, and the like. Organic powders include magnesium stearate, zinc stearate, N-acyllysine, nylon, polyethylene, polypropylene, and the like. Pigment powders include organic tar pigments and organic pigment lake pigments. Composite powders include fine-particulate titanium dioxide-coated (titanium dioxide-coated mica), fine-particulate zinc oxide-coated (titanium dioxide-coated mica), barium sulfate-coated (titanium dioxide-coated mica), silica containing titanium dioxide, and silica containing zinc oxide. Laminated powders include polyethylene terephthalate-aluminum-epoxy laminates powder, polyethylene terephthalate-polyolefin laminate films powder, polyethylene terephthalate-polymethyl methacrylate laminate films powder, and the like. One or more of these excipients may be used.

There is no particular limitation on the film-forming polymer; however, examples include water-soluble polymers and polymer emulsions which are water dispersions of water-insoluble polymers. Examples of water-soluble polymers include polyvinyl alcohol and polyvinylpyrrolidone.

Specific examples of polymer emulsions include alkyl acrylate copolymer emulsions, alkyl methacrylate copolymer emulsions, styrene-alkyl acrylate copolymer emulsions, styrene-alkyl methacrylate copolymer emulsions, vinyl acetate polymer emulsions, vinylpyrrolidone-styrene copolymer emulsions, alkyl acrylate-vinyl acetate copolymer emulsions, alkyl methacrylate-vinyl acetate copolymer emulsions, acrylic acid-alkyl acrylate copolymer emulsions, acrylic acid-alkyl methacrylate copolymer emulsions, methacrylic acid-alkyl acrylate copolymer emulsions, methacrylic acid-alkyl methacrylate copolymer emulsions, alkyl dimethicone acrylate copolymer emulsions.

The method of preparation for the water-in-oil type cosmetic composition for eyelashes of the present invention is performed according to the usual procedure. For example, in addition to the ingredients (A), (B), (C1), and (C2), any other cosmetic ingredients are mixed and stirred at a temperature above the melting point, and the container is filled.

### EXAMPLES

The invention is described below with examples; however, the invention is not limited to these examples.

Oil-in-water type cosmetic compositions for eyelashes were manufactured using the ingredients shown in Table 1. The manufactured oil-in-water type cosmetic compositions for eyelashes were evaluated for “curling effect,” “volumizing effect,” and “eyelash separation” by a panel of 10 experts.

The evaluation method was as follows

### Evaluation Methods a-c

Ten evaluators applied each sample to their own eyelashes for the following evaluation items a-c, and gave a grade on a 7-point scale from 0 to 6 based on the evaluation criteria shown in (1) below. The total of the 10 evaluators' scores was calculated, and the performance of the oil-in-water type cosmetic composition for eyelashes was judged according to the 4-step criteria shown in (2) below.

The results are shown in Table 2.

### Assessment Item

a. Curling effect
b. Volumizing effect
c. Eyelash separation
(1) Evaluation criteria
(Grade): (Rating)
6: Very good
5: Good
4: Slightly good
3: Ordinary
2: Slightly poor
1: Poor
0: Very poor
(2) Four-step criteria
(Judgment): (Total score of grade)
⊚: total score between 46 and 60 points
◯: Total score between 31 and 45 points
Δ: Total score between 16 and 30 points
x: Total score of 0 to 15 points

TABLE 1-1

| Phase | Component | | Example 1 | Example 2 | Example 3 | Example 4 | Example 5 | Example 6 | Example 7 | Example 8 |
|---|---|---|---|---|---|---|---|---|---|---|
| A | (A) | Glyceryl | 4.00 | 4.00 | 4.00 | 3.00 | 3.00 | 4.00 | — | 9.00 |
| | | Hydrogenated | | | | | | | | |
| | | Rosinate¹ | | | | | | | | |
| | (A) | Candelilla | 1.90 | 1.90 | 1.90 | 1.90 | 1.90 | 1.90 | 1.90 | 1.90 |
| | | Resin² | | | | | | | | |
| | | Trimethylsiloxy | — | — | — | — | — | — | — | — |
| | | silicate³ | | | | | | | | |
| | (B) | Carnauba Wax | 5.80 | 6.80 | 6.80 | 8.80 | 5.80 | 7.00 | 5.80 | 5.80 |
| | | (Melting Point | | | | | | | | |
| | | 80-86 °C)⁴ | | | | | | | | |
| | (B) | Bleached | 3.00 | 3.00 | 3.00 | 2.00 | 5.00 | 3.00 | 3.00 | 3.00 |
| | | Beeswax | | | | | | | | |
| | | (Melting Point | | | | | | | | |
| | | 60-70 °C, Acid | | | | | | | | |
| | | Value 17-22) ⁵ | | | | | | | | |
| | (B) | Microcrystalline | — | — | — | — | — | — | — | — |
| | | Wax (Melting | | | | | | | | |
| | | Point 86-90 °C)⁶ | | | | | | | | |
| | (C1) | (Acrylates/ | 3.00 | 3.00 | 3.00 | 3.00 | 3.00 | 3.00 | 3.00 | 3.00 |
| | | Stearyl Acrylate/ | | | | | | | | |
| | | Dimethicone | | | | | | | | |
| | | Methacrylate) | | | | | | | | |
| | | Copolymer | | | | | | | | |
| | | (Melting Point: | | | | | | | | |
| | | 25-35 °C)⁷ | | | | | | | | |
| | (C2) | Alkyl (C30-45) | 2.00 | 2.00 | 2.00 | 2.00 | 2.00 | 2.00 | 2.00 | 2.00 |
| | | Methicone | | | | | | | | |
| | | (Melting Point: | | | | | | | | |
| | | 58-65 °C)⁸ | | | | | | | | |
| | | Dimer Dilinoleyl | 1.00 | 1.00 | 1.00 | 1.00 | 1.00 | 1.00 | 1.00 | 1.00 |
| | | Dimer | | | | | | | | |
| | | Dilinoleate | | | | | | | | |
| | | Bis(behenyl/ | | | | | | | | |
| | | isostearyl/ | | | | | | | | |
| | | phytosteryl) | | | | | | | | |
| | | Dimethylpolysiloxane | 1.00 | — | — | — | b2 | 1.00 | 1.00 | 1.00 |
| | | (20 mm²/s) | | | | | | | | |
| | | Sorbitan | 1.10 | 1.10 | 1.10 | 1.10 | 1.10 | 1.10 | 1.10 | 1.10 |
| | | Monostearate | | | | | | | | |
| | | (HLB value: 4.7) | | | | | | | | |
| | | Tocopherol | 0.05 | 0.05 | 0.05 | 0.05 | 0.05 | 0.05 | 0.05 | 0.05 |
| B | | 1,3-Butylene | 5.50 | 5.50 | 5.50 | 5.50 | 5.50 | 5.50 | 5.50 | 5.50 |
| | | Glycol | | | | | | | | |
| | | 1,2-Pentanediol | 2.50 | 2.50 | 2.50 | 2.50 | 2.50 | 2.50 | 2.50 | 2.50 |
| | | Polyoxyethylene | 0.30 | 0.30 | 0.30 | 0.30 | 0.30 | 0.30 | 0.30 | 0.30 |
| | | (20) Sorbitan | | | | | | | | |
| | | Monostearate | | | | | | | | |
| | | (HLB value: | | | | | | | | |
| | | 14.9) | | | | | | | | |
| | | Polyoxyethylene | 0.60 | 0.60 | 0.60 | 0.60 | 0.60 | 0.60 | 0.60 | 0.60 |
| | | (20) Cetyl | | | | | | | | |
| | | Ether (HLB | | | | | | | | |
| | | value: 17) | | | | | | | | |
| | | Carbon Black | 0.90 | 0.90 | 0.90 | 0.90 | 0.90 | 0.90 | 0.90 | 0.60 |
| C | | Purified Water | Adjusted | Adjusted | Adjusted | Adjusted | Adjusted | Adjusted | Adjusted | Adjusted |
| | | Hydroxyethylcellulose | 0.70 | 0.70 | 0.70 | 0.70 | 0.70 | 0.70 | 0.70 | 0.70 |
| | | Gum Arabic | 1.50 | 1.50 | 1.50 | 1.50 | 1.50 | 1.50 | 1.50 | 1.50 |
| | | Polyvinylpyrrolidone | 1.00 | 1.00 | 1.00 | 1.00 | 1.00 | 1.00 | 1.00 | 1.00 |
| D | | Polyethylene | 2.80 | 2.80 | 2.80 | 2.80 | 2.80 | 2.80 | 2.80 | 2.80 |
| | | Powder | | | | | | | | |
| | | Polypropylene | 2.00 | — | 2.00 | 2.00 | 2.00 | 2.00 | 2.00 | 2.00 |
| | | Short Fibers | | | | | | | | |
| E | | (Acrylates/ | 30.00 | 30.00 | 30.00 | 30.00 | 30.00 | 30.00 | 30.00 | 30.00 |
| | | Ethylhexyl | | | | | | | | |
| | | Acrylate) | | | | | | | | |
| | | Copolymer ⁹ | | | | | | | | |
| | | (Styrene/ | 2.00 | 2.00 | 2.00 | 2.00 | 2.00 | 2.00 | 2.00 | 2.00 |
| | | Acrylates) | | | | | | | | |
| | | Copolymer¹⁰ | | | | | | | | |
| F | | Phenoxyethanol | 0.50 | 0.50 | 0.50 | 0.50 | 0.50 | 0.50 | 0.50 | 0.50 |
| | | Aminomethylpropanol | An | An | An | An | An | An | An | An |
| | | | Appro- | Appro- | Appro- | Appro- | Appro- | Appro- | Appro- | Appro- |
| | | | priate | priate | priate | priate | priate | priate | priate | priate |
| | | | Amount | Amount | Amount | Amount | Amount | Amount | Amount | Amount |
| | | (C1)/(C2) | 1.50 | 1.50 | 1.50 | 1.50 | 1.50 | 1.50 | 1.50 | 1.50 |
| | | ((A) + (B))/(C) | 2.94 | 3.14 | 3.14 | 3.14 | 3.14 | 3.18 | 2.14 | 3.94 |

TABLE 1-2

| | | | Comparative | Comparative | Comparative | Comparative | Comparative | Comparative | Comparative |
|---|---|---|---|---|---|---|---|---|---|
| Phase | Component | | Example 1 | Example 2 | Example 3 | Example 4 | Example 5 | Example 6 | Example 7 |
| A | (A) | Glyceryl | 4.00 | 4.00 | 4.00 | 4.00 | — | — | 8.00 |
| | | Hydrogenated | | | | | | | |
| | | Rosinate¹ | | | | | | | |
| | (A) | Candelilla | 1.90 | 1.90 | 1.90 | 1.90 | — | — | 3.80 |
| | | Resin² | | | | | | | |
| | | Trimethylsiloxy | — | — | — | — | 11.80 | — | — |
| | | silicate³ | | | | | | | |
| | (B) | Carnauba Wax | 5.80 | 5.80 | 5.80 | — | 5.80 | 9.80 | 1.00 |
| | | (Melting Point | | | | | | | |
| | | 80-86 °C)⁴ | | | | | | | |
| | (B) | Bleached | 3.00 | 3.00 | 3.00 | 3.00 | 3.00 | 4.80 | 1.00 |
| | | Beeswax | | | | | | | |
| | | (Melting Point | | | | | | | |
| | | 60-70 °C, Acid | | | | | | | |
| | | Value 17-22) ⁵ | | | | | | | |
| | (B) | Microcrystalline | — | — | — | 5.80 | — | — | — |
| | | Wax (Melting | | | | | | | |
| | | Point 86-90 °C)⁶ | | | | | | | |
| | (C1) | (Acrylates/ | 1.00 | 5.00 | — | 1.00 | 3.00 | 3.00 | 3.00 |
| | | Stearyl Acrylate/ | | | | | | | |
| | | Dimethicone | | | | | | | |
| | | Methacrylate) | | | | | | | |
| | | Copolymer | | | | | | | |
| | | (Melting Point: | | | | | | | |
| | | 25-35 °C)⁷ | | | | | | | |
| | (C2) | Alkyl (C30-45) | 4.00 | — | — | 1.50 | 2.00 | 2.00 | 2.00 |
| | | Methicone | | | | | | | |
| | | (Melting Point: | | | | | | | |
| | | 58-65 °C)⁸ | | | | | | | |
| | | Dimer Dilinoleyl | 1.00 | 1.00 | 1.00 | 1.00 | 1.00 | 1.00 | 1.00 |
| | | Dimer | | | | | | | |
| | | Dilinoleate | | | | | | | |
| | | Bis(behenyl/ | | | | | | | |
| | | isostearyl/ | | | | | | | |
| | | phytosteryl) | | | | | | | |
| | | Dimethylpolysiloxane | 1.00 | 1.00 | 1.00 | 1.00 | 1.00 | 1.00 | 1.00 |
| | | (20 mm²/s) | | | | | | | |
| | | Sorbitan | 1.10 | 1.10 | 1.10 | 1.10 | 1.10 | 1.10 | 1.10 |
| | | Monostearate | | | | | | | |
| | | (HLB value: 4.7) | | | | | | | |
| | | Tocopherol | 0.05 | 0.05 | 0.05 | 0.05 | 0.05 | 0.05 | 0.05 |
| B | | 1,3-Butylene | 5.50 | 5.50 | 5.50 | 5.50 | 5.50 | 5.50 | 5.50 |
| | | Glycol | | | | | | | |
| | | 1,2-Pentanediol | 2.50 | 2.50 | 2.50 | 2.50 | 2.50 | 2.50 | 2.50 |
| | | Polyoxyethylene | 0.30 | 0.30 | 0.30 | 0.30 | 0.30 | 0.30 | 0.30 |
| | | (20) Sorbitan | | | | | | | |
| | | Monostearate | | | | | | | |
| | | (HLB value: | | | | | | | |
| | | 14.9) | | | | | | | |
| | | Polyoxyethylene | 0.60 | 0.60 | 0.60 | 0.60 | 0.60 | 0.60 | 0.60 |
| | | (20) Cetyl | | | | | | | |
| | | Ether (HLB | | | | | | | |
| | | value: 17) | | | | | | | |
| | | Carbon Black | 0.90 | 0.90 | 0.90 | 0.90 | 0.90 | 0.90 | 0.90 |
| C | | Purified Water | Adjusted | Adjusted | Adjusted | Adjusted | Adjusted | Adjusted | Adjusted |
| | | Hydroxyethylcellulose | 0.70 | 0.70 | 0.70 | 0.70 | 0.70 | 0.70 | 0.70 |
| | | Gum Arabic | 1.50 | 1.50 | 1.50 | 1.50 | 1.50 | 1.50 | 1.50 |
| | | Polyvinylpyrrolidone | 1.00 | 1.00 | 1.00 | 1.00 | 1.00 | 1.00 | 1.00 |
| D | | Polyethylene | 2.80 | 2.80 | 2.80 | 2.80 | 2.80 | 2.80 | 2.80 |
| | | Powder | | | | | | | |
| | | Polypropylene | 2.00 | 2.00 | 2.00 | 2.00 | 2.00 | 2.00 | 2.00 |
| | | Short Fibers | | | | | | | |
| E | | (Acrylates/ | 30.00 | 30.00 | 30.00 | 30.00 | 30.00 | 30.00 | 30.00 |
| | | Ethylhexyl | | | | | | | |
| | | Acrylate) | | | | | | | |
| | | Copolymer ⁹ | | | | | | | |
| | | (Styrene/ | 2.00 | 2.00 | 2.00 | 2.00 | 2.00 | 2.00 | 2.00 |
| | | Acrylates) | | | | | | | |
| | | Copolymer¹⁰ | | | | | | | |
| F | | Phenoxyethanol | 0.50 | 0.50 | 0.50 | 0.50 | 0.50 | 0.50 | 0.50 |
| | | Aminomethylpropanol | An | An | An | An | An | An | An |
| | | | Appropriate | Appropriate | Appropriate | Appropriate | Appropriate | Appropriate | Appropriate |
| | | | Amount | Amount | Amount | Amount | Amount | Amount | Amount |
| | | (C1)/(C2) | 0.25 | — | — | 0.66667 | 1.50 | 1.50 | 1.50 |
| | | ((A) + (B))/(C) | 2.94 | 2.94 | — | 5.88 | 1.76 | 2.92 | 2.76 |

TABLE 2

| | | Example 1 | Example 2 | Example 3 | Example 4 | Example 5 | Example 6 | Example 7 | Example 8 |
|---|---|---|---|---|---|---|---|---|---|
| Evaluation | Curling | ⊚ | ⊚ | ⊚ | ⊚ | ◯ | ⊚ | ◯ | ⊚ |
| | Effect | | | | | | | | |
| | Volumizing | ⊚ | ⊚ | ⊚ | ⊚ | ⊚ | ⊚ | ◯ | ⊚ |
| | Effect | | | | | | | | |
| | Lash | ⊚ | ◯ | ◯ | ◯ | ◯ | ◯ | ⊚ | ◯ |
| | Separation | | | | | | | | |

| | | Compar- | Compar- | Compar- | Compar- | Compar- | Compar- | Compar- |
|---|---|---|---|---|---|---|---|---|
| | | ative | ative | ative | ative | ative | ative | ative |
| | | Example 1 | Example 2 | Example 3 | Example 4 | Example 5 | Example 6 | Example 7 |
| Evaluation | Curling | ◯ | ◯ | ⊚ | Δ | ◯ | ◯ | ◯ |
| | Effect | | | | | | | |
| | Volumizing | Δ | ⊚ | ◯ | Δ | Δ | ◯ | Δ |
| | Effect | | | | | | | |
| | Lash | ⊚ | Δ | Δ | Δ | Δ | Δ | ⊚ |
| | Separation | | | | | | | |

¹ PINECRYSTAL KE-311 (manufactured by Arakawa Chemical Industries, Ltd.)
² Candelilla Resin E-1 (manufactured by Nippon Natural Products Co., Ltd.)
³ KF-7312J (Shin-Etsu Chemical Co., Ltd.): A 50/50 mixture of trimethylsiloxysilicate and decamethylcyclopentasiloxane (by mass).
⁴ Refined Carnauba Wax No. 1 (manufactured by Cerarica Noda Co., Ltd.)
⁵ WHITE BEES WAX (manufactured by Miki Chemical & Co., Ltd.): Wax with an acid value of 17-22
⁶ Hi-Mic-1090 (manufactured by Nippon Seiro Co., Ltd.)
⁷ KP-561P (manufactured by Shin-Etsu Chemical Co., Ltd.)
⁸ Gransil VX-404 (manufactured by Grant Industries, Inc.)
⁹ Daitosol 5000SJ (manufactured by Daito Kasei Kogyo Co., Ltd.): 50% solids
¹⁰ Daitosol 5000STY (manufactured by Daito Kasei Kogyo Co., Ltd.): solids concentration 50%

As shown in Tables 1 and 2, it was confirmed that the oil-in-water type cosmetic compositions for eyelashes (mascaras) of Examples 1-8 were superior in curling effect, volumizing effect, and eyelash separation property compared to the oil-in-water type cosmetic compositions for eyelashes of Comparative Examples 1-5. On the other hand, the volumizing effect was insufficient for the oil-in-water type cosmetic compositions for eyelashes of Comparative Examples 1 and 4, in which the ratio (mass ratio) (C1)/(C2) of component (C1) to component (C2) was less than 1, and the eyelash separation property of Comparative Example 2, which does not contain component (C2), was unsatisfactory. In addition, the oil-in-water type cosmetic composition for eyelashes of Comparative Example 3, which contained neither component (C1) nor component (C2), was not satisfactory with respect to eyelash separation. The oil-in-water type cosmetic composition for eyelashes of Comparative Example 6, which contained no component (A), was not satisfactory with respect to eyelash separation. The oil-in-water type cosmetic composition for eyelashes of Comparative Example 5, which contained trimethylsiloxysilicate in place of component (A), was unsatisfactory with respect to volumizing effect and eyelash separation. The oil-in-water type cosmetic composition for eyelashes of Comparative Example 7, which contained a small amount of component (B), exhibited an inferior volumizing effect.

1. An oil-in-water type cosmetic composition for eyelashes, comprising:
(A) 1 to 12% by mass of an oil-soluble resin;
(B) 3 to 10% by mass of a solid oil having a melting point of 50 to 120 °C;
(C1) a low-melting-point silicone wax having a melting point in the range of 10 °C to less than 50 °C; and
(C2) a high-melting-point silicone wax having a melting point of 50 to 110 °C, wherein:
the total amount of component (C1) and component (C2) is in the range of 1 to 10% by mass,
the mass ratio of component (C1) to component (C2), (C1)/(C2), is in the range of 1 to 5, and
the mass ratio of the total amount of component (A) and component (B) to the total amount of component (C1) and component (C2), [(A)+(B)]/[(C1)+(C2)], is 2 or more.

2. The cosmetic composition according to claim 1, wherein the mass ratio of the total amount of components (A) and (B) to the total amount of components (C1) and (C2), [(A)+(B)]/[(C1)+(C2)], is in the range of 2.5 to 4.5.

3. The cosmetic composition according to claim 1, wherein the solid oil of component (B) having a melting point of 50 to 120 °C comprises carnauba wax.

4. The cosmetic composition according to claim 3, wherein the amount of carnauba wax is more than 50% by mass of the total amount of the solid oil.
